(19) United States
(12) Patent Application Publication
(10) Pub. No.: US 2001/0053917 A1
(43) Pub. Date: Dec. 20, 2001

(54) Non-plane cut microkeratome and method of performing non-plane keratotomy

(76) Inventors: Chwen-Yih Lin, Banchiau City (TW); Jui-Fang Tsai, Banchiau City (TW)

Correspondence Address: BACON & THOMAS, PLLC, 625 SLATERS LANE, FOURTH FLOOR, ALEXANDRIA, VA, 22314

(21) Appl. No.: 09/880,802

(22) Filed: Jun. 15, 2001

(30) Foreign Application Priority Data
Jun. 16, 2000 (TW) 089111789

(51) Int. Cl. A61F 9/00

(52) U.S. Cl. 606/166

(57) ABSTRACT

A microkeratome and method for performing lamellar keratoplasty is disclosed. The microkeratome consist of a cutting blade with a curved sharp edge. In surgery, a programmed curved guide rail is utilized to guide the cutting blade to move along a programmed curved path so that achieve a near spherical curved cutting pathway so as to cut the cornea in near its original un-flattened normal position. The microkeratome for performing lamellar keratoplasty includes a vacuum suction ring with guide rails, a cutting head assembly with a cambered cutting blade and curved front press and curved rear press. By controlling the internal components of the tool so that the vacuum suction ring can fix an eyeball. The microkeratome cuts cornea along a preset path of tracks, since the cutting blade is curved and the front press and rear press are also curved surfaces so that the cutting path is formed with a curved surface, thereby the microkeratome can cut in a path that keep the cornea remain in an un-flattened original position, thus reduce the deformity that in prior art plane cut will induce.

### BACKGROUND OF THE INVENTION

1. Field of the Invention

The present invention relates to medical instruments and methods for performing lamellar keratoplasty, in particular, to the use of a refractive laser in conjunction with an automated lamellar keratoplasty, so-called LASIK (laser-in-situ keratoplasty). The present invention teaches a new method and a novel apparatus for improving the creation of the lamellar corneal flap.

2. Description of Related Art

LASIK (laser-in-situ keratoplasty) is a corneal surgery for correction of refractive errors such as myopia, hyperopia and astigmatism. Aspects of the procedure are disclosed and claimed in U.S. Pat. No. 4,840,175 to Peyman et al and U.S. Pat. No. 4,903,695, to Warner et al. The refractive procedure requires the creation of a lamellar corneal flap. Many mechanical devices have been described to create the lamellar flap. These devices, generally known as “microkeratome”, differ principally in how they drive the blade and how they flatten the cornea during the lamellar cut. For example, U.S. Pat. No. 5,586,980, to Kremer et al discloses a microkeratome device.

The prior art microkeratome devices all need to flatten the cornea to an extreme plane position while cutting is performed. Some of the design as disclosed in U.S. Pat. Nos. 5,591,174, 6,228,099, 6,071,293, 6,197,038, 6,126,668, 5,997,559, 6,165,189, 6,022,365, 5,507,759, 6,183,488, RE035421, U.S. Pat. No. 5,133,726 the cutting head is going straightforward while they are cutting the cornea; the cutting paths of the cutting knives are in a plane fashion. Some of the design as disclosed in U.S. Pat. Nos. 5,980,583, 6,051,009, 6,007,553, 5,624,456, 5,586,980 the cutting head are driven along sweeping circular rail or forward along a pivot center, despite the cutting head move in a arcuate path the cutting plane are also in a plane fashion, the cornea need to be applanated to a plane position before the cut begin. These prior art use plane shape knives even driven by gear or wire to cut through plane fashion path, they need to flatten the convex cornea to a extreme plane position before cut begin. The disadvantage of the prior art is that the cornea is deformed from its normal position to extreme plane position. The cornea tissue is a elastic material, the shape change is very distinct from a convex shape to a plane shape, especial in the vertex region. The internal deformity of the cornea tissue is great and unpredictable, they lead to cracking-like striates and polymorphism of micro-deformation lead to the result of irregular astigmatism which is difficult to be correct. Despite some of design of prior art as in U.S. Pat. No. 6,007,553 to Carriazo et al use concave or convex flatten plag to deform cornea while the cutting is proceed, the cutting knife is generally cutting along plane path, the deformation is still very huge, hence lead to unpredictable uneven result in creating the flaps.

Another design as U.S. Pat. No. 5,643,299, to Scott Bair use liquid jet for cutting the cornea, the applanation of cornea is also very significant and the cutting of the liquid jet is generally in linear or plane fashion. The deformation of the cornea due to the stretch from its original normal position to the cutting position is great. The uneven internal deformation lead to significant imperfect corneal flap formation.

### SUMMARY OF THE INVENTION

In response to the shortenings described herein, it is an object of the present invention to provide an improved microkeratome wherein the cutting path of the cutting knife is generally along the corneal original normal position, and left the cornea only be deformed in minimum extent.

It is a further object of the present invention to provide a microkeratome wherein the cutting knife is in curved bending fashion in order to oscillate and forward along a generally near spherical cutting path.

It is a further object of the present invention to provide a cutting head with curved guide slit for which the curved bending cutting knife can oscillate within the guide slit to create oscillating motion for cutting of cornea. And the curved bending knife oscillates in a curved back and forward stroke.

It is a further object of the present invention to provide a predetermined curved guiding rail wherein the cutting head with the bended cutting knife can move along it to create a generally near spherical cutting path to keep the cornea in near its original normal position while it is cut.

It is a further object of the present invention to provide an anterior guiding plate just in front of the cutting edge wherein it's curvature of lower surface is close to the average of corneal curvature, so that it can press the cornea to it's original position while the eyeball is fixed with vacuum suction ring and the cornea protrude upward while the vacuum is activated.

It is a further object of the present invention to provide a posterior guiding plate just after the cutting edge, wherein it's curvature of lower surface is close to the average of corneal curvature, so that it can press the cornea to its original position while the eyeball is fixed with vacuum suction ring and the cornea protrude upward while cutting is performed.

It is a further object of the present invention to provide a vacuum eyeball fixation ring wherein it's central aperture only let the corneal flap portion and the extra flap hinge portion to be exposed, so that the cutting head can only cut along the designed flap position of cornea, and by limitation of movement of the cutting head, the extra hinge portion will be left intact.

The various objects and advantages of the present invention will be more readily understood from the following detailed description when read in conjunction with the appended drawing.

### BRIEF DESCRIPTION OF THE DRAWINGS

FIG. 1 is a schematic view showing the operation of cutting a cornea in a prior art eye surgery.

FIG. 2 is a perspective view of the cambered surface of the microkeratome for performing lamellar keratoplasty of the present invention.

FIG. 2A is a exploded perspective view of the present invention.

FIG. 2B is a perspective view of the present invention.

FIG. 3 shows an embodiment of the microkeratome for performing lamellar keratoplasty in the present invention.

FIG. 4 is a schematic view showing the operation of cutting cornea in the present invention.

FIG. 5 is a schematic view of the tracks of the microkeratome for performing lamellar keratoplasty in the present invention.

FIG. 6 is a schematic view of laser treating in the microkeratome for performing lamellar keratoplasty according to the present invention.

FIG. 7 shows another embodiment of the microkeratome for performing lamellar keratoplasty according to the present invention.

FIG. 8 is a upper view for showing the extra expanding region of the guide set of the microkeratome for performing lamellar keratoplasty in the present invention.

### DETAILED DESCRIPTION OF THE INVENTION

In order that those skilled in the art can further understand the present invention, a description will be described in the following in details. However, these descriptions and the appended drawings are only used to cause those skilled in the art to understand the objects, features, and characteristics of the present invention, but not to be used to confine the scope and spirit of the present invention defined in the appended claims.

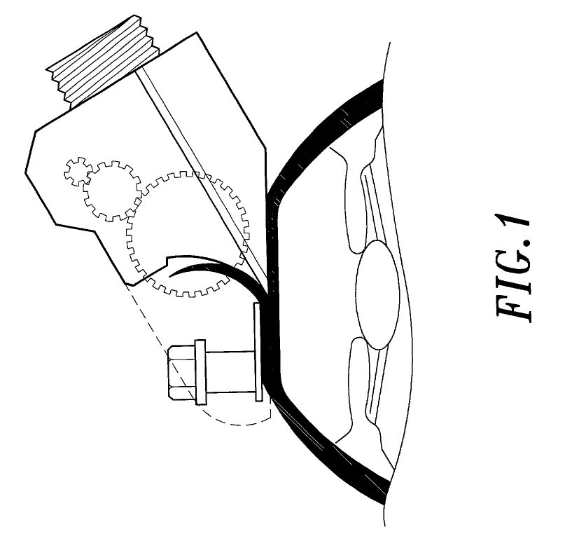

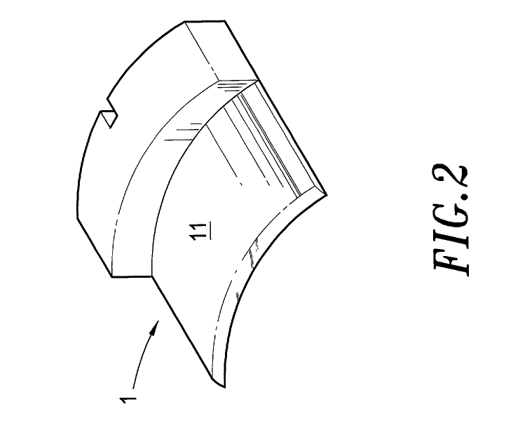

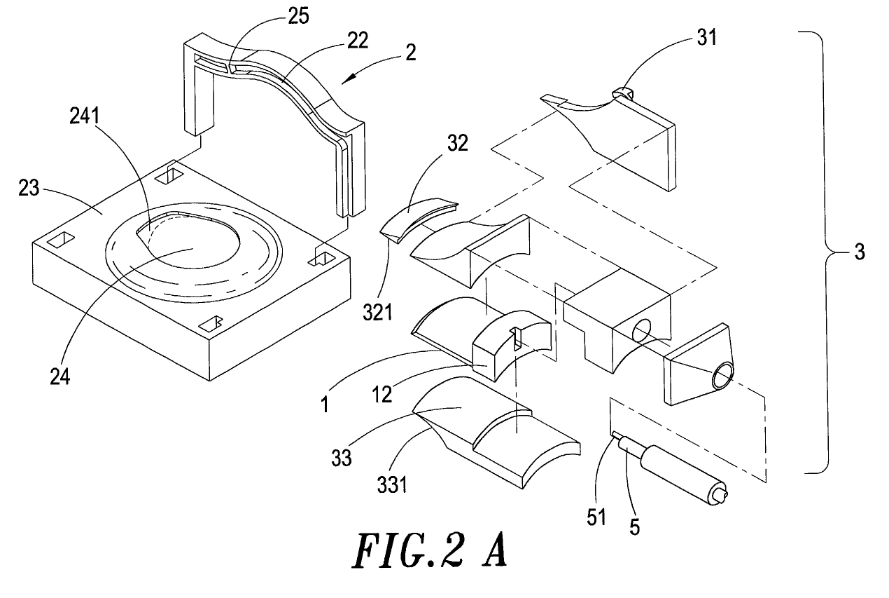

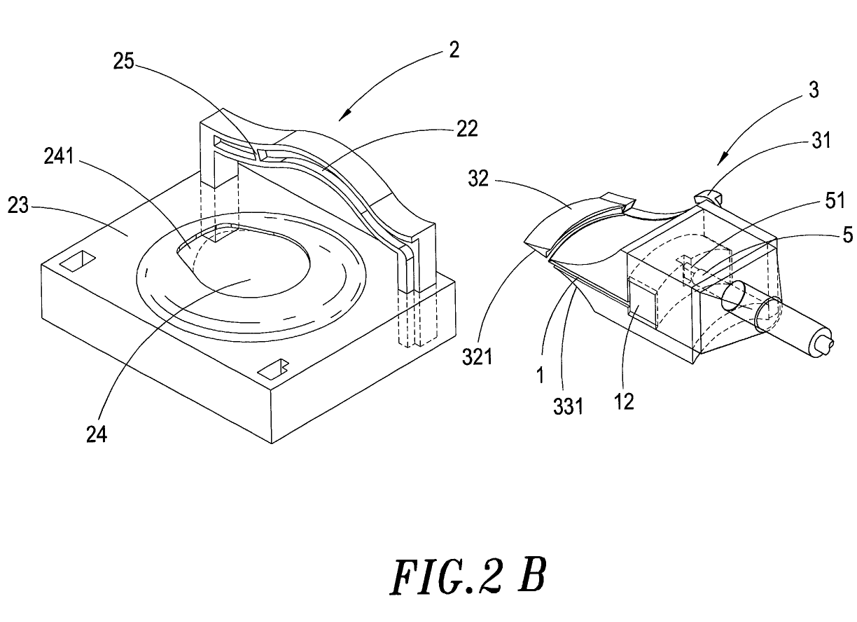

Referring to FIGS. 2, the microkeratome for performing lamellar keratoplasty according to the present invention is illustrated. The microkeratome consist of a cambered cutting blade 1 with a curved sharp edge. In surgery, the arc of the track is utilized to match a knife without needing to flat an eyeball so as to achieve the object of performing lamellar keratoplasty. Since the prior art microkeratome has a flat knife surface (referring to FIG. 1), in cutting a cornea, the cornea must be smoothened to plane position, but the normal position of cornea is a convex fashion it will induce a dramatic deformity in cornea tissue in order to render to extreme plane position, Thus the irregularity in the cornea tissue will result in an un-uniform flap formation.

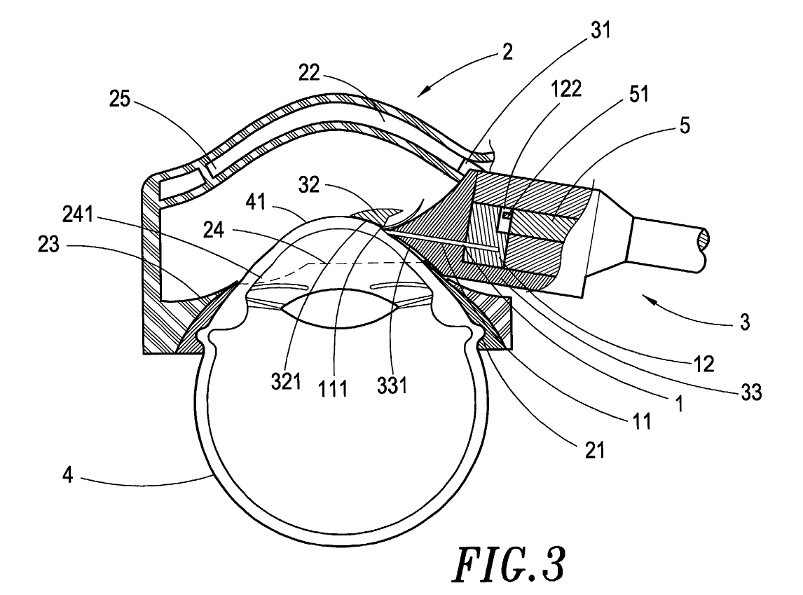

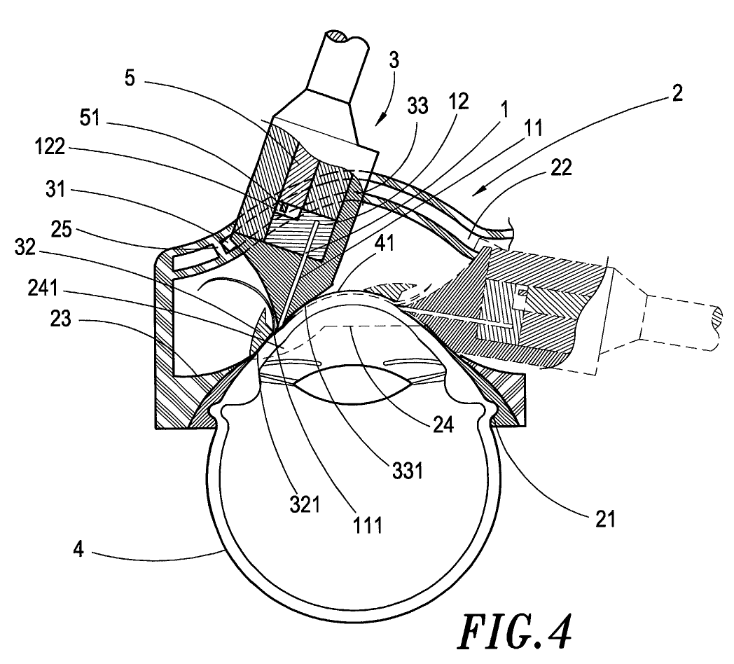

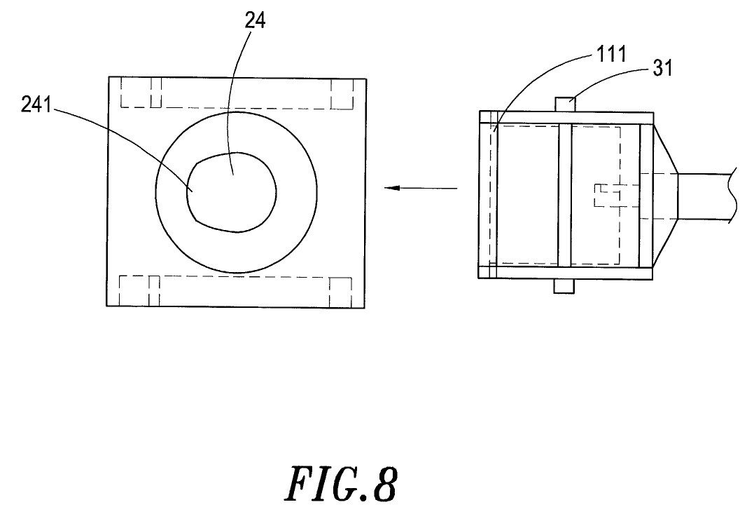

Referring to FIGS. 3 and 4, the microkeratome of the present invention has two parts. One is a suction ring guide set 2, and the other is a cutting head set 3. In the present invention, a vacuum suction ring 21 is firstly used to position an eyeball so that the cornea 41 protrudes forwards to a predetermined cutting position and exposed only part to be cut. Then cutting blade 1 cuts the exposed portion along a preset path. The guide pin 31 of the cutting head set 3 moves forwards along a path of the track 22 so that the cutting path is kept in the programmed curved fashion, since the cutting blade 1 is also a cambered knife which oscillated transverse to the forward direction of cutting head, Thus, the cornea 41 can be cut without hugely deformed to plane position, The cornea is cut in nearly spherical fashion and it is very close to the original normal position of cornea itself, Therefore, the deformity in cutting a cornea 41 is greatly reduced. The lower plate 21 of the suction ring guide set 2 is an eyeball suction ring which is installed with a via hole 24. The eyeball can be aligned to this via hole 24 for performing lamellar keratoplasty. Furthermore, an extra portion 241 in one side of the via hole 24 serves to leave the hinge root portion of the cornea to be intact(refer to FIG. 8, FIG. 2A, FIG. 2B for further reference). This extra portion 241 of the via hole in concert with the stop point 25 of the suction ring guide set 2 can protect the hinge of flap from been cut through. The two tracks 22 at two sides cause that the cutting blade 1 of the microkeratome may move forwards along a programmable path assigned within the track 22. A stop point 25 is installed at the track 22 which is so arranged that the cutting head 3 slides along the track 22 to the stop point 25 will produce a cutting path just near the corneal normal un-flattened position. In the cutting head 3, the cutting blade 1 will oscillated via a eccentric driving rod 51 to produce a back and forward motion in the direction transverse to the advance path of the cutting head 3. Two sides of the cutting head 3 have guide pins 31. The pins 31 are placed into the tracks 22 so that as the cutting head 3 moves forwards steadily along the programmed tracks 22 can cut the cornea in a designed pathway. A front press 32 with a spherical inferior surface is installed at the front side of the cutting head 3. As the cornea 41 is cut, the front press 32 which consist of a spherical inferior surface 321 presses the eyeball to near its normal position to reduce the great distortion induced by deformation of cornea. The gap 111 in the cutting head 3 is used to determine the thickness of the cornea 41 necessary to be cut. Then a rear press 33 which consist of a spherical inferior surface 331 is used to retain the cornea to keep in its near normal position while cutting is performing.

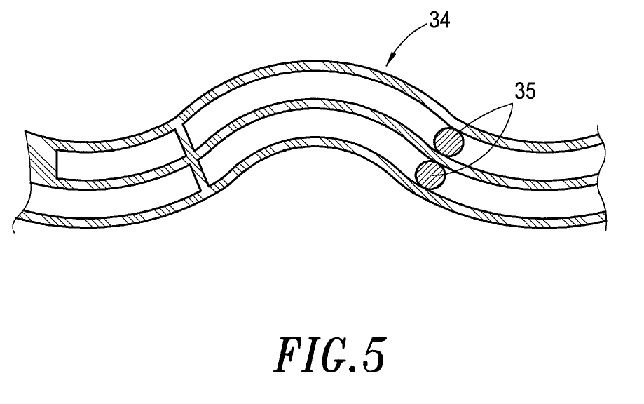

Referring to FIG. 5, the track 22 is made as a double grooves guide 34 and two round posts 35 are used to replace the pins 31. The direction of the cutting blade 1 in the cutting head 3 is maintained a fixed angle with the line connect the centers of the two posts. This arrangement can maintain the relative cutting angle of the cutting blade on the cornea.

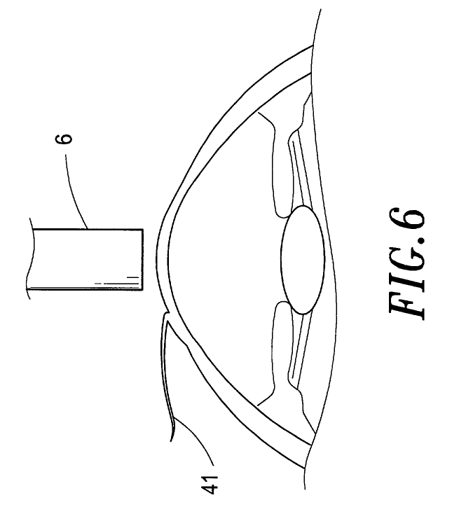

The power for driving the cutting blade 1 from the rear end is from a driving shaft 5. The driving shaft 5 has a front eccentric shaft 51 to be placed into a slot 122 which located in the rear side of the knife seat 12. When the driving shaft 5 moves forwards and the eccentric shaft 51 rotates, the knife seat 12 will be driven to move in transverse forward and backward strokes and since the pins 31 move along the track 22, so that the cutting edge in the front end of the cutting blade 1 cuts the cornea 41 along a preset path defined by the track 22. After the cornea 41 is cut by a cambered cutting blade 1 (referring to FIG. 6), the stroma of the cornea is opened for laser 6 treating.

Referring to FIG. 8, and FIG. 2A and FIG. 2B, in the present invention, in order to assign the area of cornea to be cut and leave the flap hinge region to be intact, There is a central via hole 24 and an extra exposure region 241 in the suction ring guide set 2, The central via hole 24 serves as a exposure region for the cornea for cut. The cornea area outsides the central via hole 24 will not cut since they are protected from the suction ring itself. The extra exposure region 241 is designed to determine the cornea flap hinge region. Since the cutting blade 1 will be stopped while the end point 25 reached by the guide pin 31 and the cutting blade 1 will stopped before it cut through the extra region 241, so that the extra region 241 can determine the width of the hinge. By designing a different size of extra exposure region 241 we can change the width of the hinge of the cornea flap.

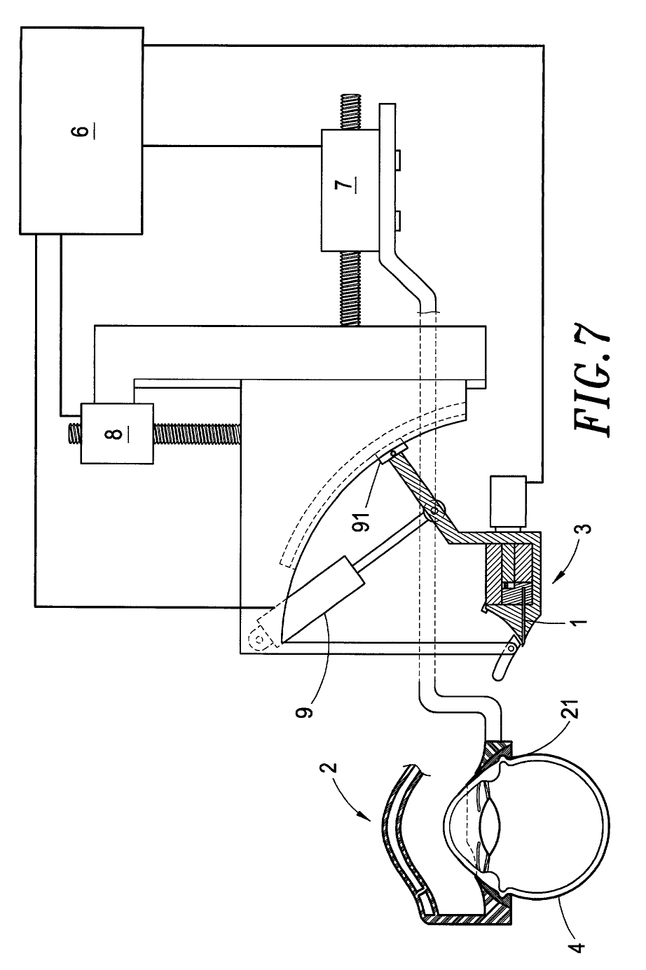

Other than using the hand to precisely control the surgery, the lamellar keratoplasty can be positioned automatically. Referring to FIG. 7, a programmable path control unit 6 includes a horizontal forwards moving driver 7, a vertical controller 8, and a knife angle controller 9. After the eyeball is fixed by a vacuum suction ring 21, the knife angle controller 9 is used to adjust the position of a sliding block 91 to change the cut angle to a position for cutting the cornea. Then the cutting head is adjusted to an optimum position by the horizontal forwards moving driver and vertical controller. When the cornea is cut, the horizontal forwards moving driver and vertical controller will change the position of the cutting head 3 by the programmed step. Since the cutting path is entirely programmable, by utilizing the cambered cutting blade 1, the cutting pathway of the cornea can be kept to be spherical fashion. The most important is that the cornea can be cut with the sharp knife in its original normal un-flattened position so that the deformity in cutting reduced greatly.

Therefore, the microkeratome for performing lamellar keratoplasty of the present invention has the following advantages:

1. By the cambered cutting knife of the microkeratome, and the curved surfaces of the front press and the rear press, a spherical surface cut is formed so that the deformity in cutting the cornea thus reduced greatly.

2. The via hole at the vacuum suction ring is used to determine the protruding portion of an eyeball so that the determine the cornea area to be cut.

3. The extra exposure region of the vacuum suction ring can determine the width of the hinge of the cornea flap.

The present invention are thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A microkeratome system for performing lamellar keratoplasty comprising:
a cutting head set having a cutting blade with cambered shape, a front press has a spherical inferior surface being formed at a front guide portion of the cutting head set; a rear press has a spherical inferior surface just located after the cutting edge of the cutting blade; two sides at an outer edge thereof having guide pins; and driving shaft for driving the knife to oscillate; and a base guide set being a hollow suction ring and installed with a via hole for aligning and positioning an eyeball for surgery, two sides thereof being installed with tracks so that the cutting head moves along a programmable path;
by above components, the cutting head with cambered shape knife moves along the tracks of the guiding set so that the cut path is a curved surface, in surgery, the cornea is unnecessary to be flattened to plane shape so that the cornea is cut in near its original normal position and the corneal tissue deformity in cutting is reduced.

2. The microkeratome for performing lamellar keratoplasty as claimed in claim 1, wherein the track is a smooth track.

3. The microkeratome for performing lamellar keratoplasty as claimed in claim 1, wherein each track is a toothed gear guide track.

4. The microkeratome for performing lamellar keratoplasty as claimed in claim 1, wherein a moving path of the track is a cambered path or a plurality of cambered paths, or combination of multiple curved paths.

5. The microkeratome for performing lamellar keratoplasty as claimed in claim 1, wherein in the via hole of the base guide set, a vacuum suction ring is used to position the eyeball, and then an extra exposure region serves to determine the size of the hinge at the root portion of the cornea flap, so that the entire exposed portion can be used to determine the size of the cornea flap to be cut and the width of the hinge to be leave intact.

6. The microkeratome for performing lamellar keratoplasty as claimed in claim 1, wherein when a cornea is cut, a horizontal moving controller and a vertical level controller and a cutting angle controller as well as a programmable computerized control unit are utilized for driving the cutting head with cambered shape cutting blade to move with the programmed pathway in order to cut the cornea in its normal un-flattened position.

7. The microkeratome for performing lamellar keratoplasty as claimed in claim 1, wherein the contact surface of the front press with the cornea is a curved surface.

8. The microkeratome for performing lamellar keratoplasty as claimed in claim 1, wherein the contact surface of the rear press with the cornea is a curved surface.

9. The microkeratome for performing lamellar keratoplasty as claimed in claim 1, wherein the track is a double guide groove and the guide pin is formed by two round posts.

10. A method for keratomileusis ophthalmic surgery, comprising the steps of: fixing a suction ring guide set having a suction ring and at least one programmed curved guide rail to an ocular globe about the globe's cornea so that the cornea extends through a suction ring aperture which consist an extra exposure region in one side, so that only protrude cornea can be cut and the un-exposed cornea remain shield, applying a cutting head having a cambered cutting blade along at least a predetermined arcuate guide rail to advance from an initial position to an end position to form a near spherical curved cutting pathway so as to cut the cornea in near its un-flattened original normal position.

11. The method of claim 10, wherein the motion of the advance of the cutting head is controlled with a programmable computerized system having a horizontal motion controller for horizontal movement of the cutting head, a vertical motion controller for the vertical movement of the cutting head, a cutting angle controller for the adjustment of the cutting angle of the cutting blade.
